(19) United States
(12) Patent Application Publication
(10) Pub. No.: US 2026/0041890 A1
(43) Pub. Date: Feb. 12, 2026

(54) CATHETER STABILIZATION SYSTEM AND CATHETER STABILIZATION DEVICE

(71) Applicants: Gilbert James McLaughlin, Tucson, AZ (US); David Michael Kriesel, Rancho Santa Fe, CA (US)

(72) Inventors: Gilbert James McLaughlin, Tucson, AZ (US); David Michael Kriesel, Rancho Santa Fe, CA (US)

(21) Appl. No.: 18/795,779

(22) Filed: Aug. 6, 2024

(51) Int. Cl. A61M 25/02

(52) U.S. Cl. CPC A61M 25/02; A61M 2025/0206

(57) ABSTRACT

A catheter stabilization device of a catheter stabilization system includes a base having a top surface and a bottom surface, a first arm having a first inner surface and a second arm having a second inner surface, the first arm and the second arm each extending upwardly from the top surface of the base, the first inner surface and the second inner surface defining between them a space for receiving a valve or sample port for a catheter, the valve or sample port including inlet and outlet components and an enlarged portion between the inlet and outlet components, the first inner surface and the second inner surface further defining between them inlet and outlet openings at ends of the space for the inlet and outlet components of the valve or sample port, the inlet and outlet openings being narrower than a widest portion of the space.

### BACKGROUND AND SUMMARY

The present invention relates generally to devices and systems for stabilizing catheters relative to a user.

Catheters such as Foley catheters typically involve a balloon end, an inflation line, and a drainage line. The drainage line typically leads to a valve or sample port which, in turn is connected to a drainage bag. When opening or closing the valve when removing or emptying the drainage bag or taking samples from a sample port, the drainage line can be jostled, twisted, or kinked, which can cause substantial pain to the user. It is desirable to provide a system and device to reduce the possibility of a catheter drainage line being handled in a manner that might cause discomfort to the user.

In accordance with an aspect of the present invention, a catheter stabilization device comprises a base having a top surface and a bottom surface, a first arm having a first inner surface and a second arm having a second inner surface, the first arm and the second arm each extending upwardly from the top surface of the base, the first inner surface and the second inner surface defining between them a space for receiving a valve or sample port for a catheter, the valve or sample port including inlet and outlet components and an enlarged portion between the inlet and outlet components, the first inner surface and the second inner surface further defining between them inlet and outlet openings at ends of the space for the inlet and outlet components of the valve or sample port, the inlet and outlet openings being narrower than a widest portion of the space.

In accordance with another aspect of the present invention, a catheter stabilization system comprises a catheter comprising a drainage port, a valve or sample port attached to the drainage port, the valve or sample port including inlet and outlet components and an enlarged portion between the inlet and outlet components, and a catheter stabilization device, the catheter stabilization device comprising a base having a top surface and a bottom surface, and a first arm having a first inner surface and a second arm having a second inner surface, the first arm and the second arm each extending upwardly from the top surface of the base, the first inner surface and the second inner surface defining between them a space of sufficient size to receiving the enlarged portion of the valve or sample port, the first inner surface and the second inner surface further defining between them inlet and outlet openings at ends of the space for the inlet and outlet components of the valve or sample port, the inlet and outlet openings being narrower than a widest portion of the space.

### BRIEF DESCRIPTION OF THE DRAWINGS

The features and advantages of the present invention are well understood by reading the following detailed description in conjunction with the drawings in which like numerals indicate similar elements and in which:

FIG. 1 is a top view of a catheter stabilization system, the system including a catheter stabilization device, according to an aspect of the present invention;

FIG. 2 is a perspective view of a catheter stabilization device according to an aspect of the present invention;

FIG. 3 is a side view of the catheter stabilization device of FIG. 2; and

FIG. 4 is a top view of the catheter stabilization device of FIG. 2.

### DETAILED DESCRIPTION

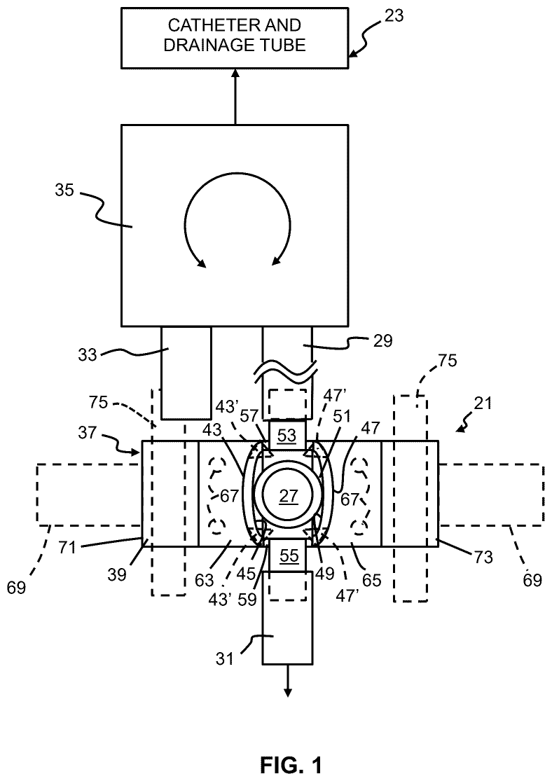

FIG. 1 shows a catheter stabilization system including a device 21 for stabilizing a catheter 23 according to an aspect of the present invention. The stabilization device 21 is shown in FIG. 1 with portions of the catheter 23 including a valve or sample port 27, a portion of an upstream drainage line 29 from the catheter to the valve or sample port, and a portion of a downstream drainage line 31 from the valve or sample port, usually leading to a drainage bag. The catheter also typically includes an inflation line 33. The upstream drainage line 29 and the inflation line 33 may be but are not necessarily secured relative to each other via another stabilization device such as a StatLock Foley Stabilization Device 35 which may be of a type that permits the lines to pivot through at least a limited arc about an axis on the StatLock Device.

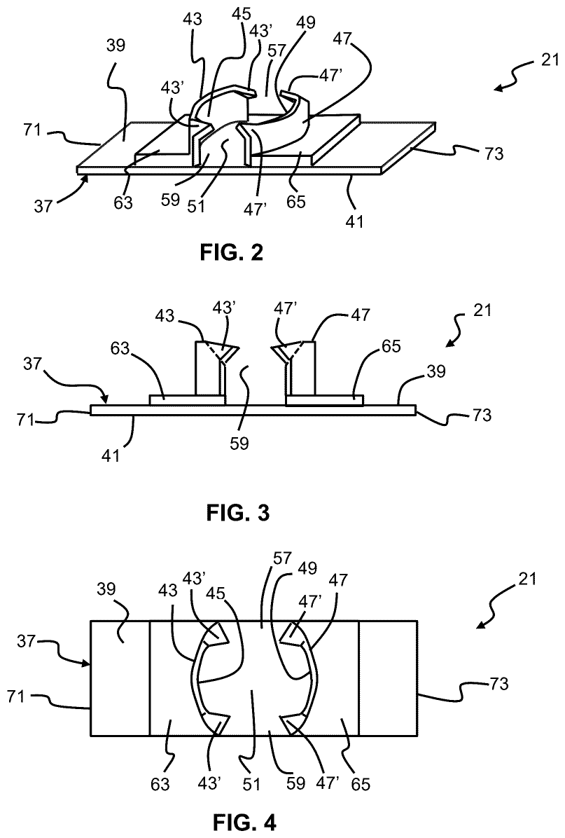

The stabilization device 21 according to an aspect of the present invention, shown without catheter portions in FIGS. 2-4, is typically but not necessarily a plastic component of a material suitable for medical use and skin contact and comprises a base 37 having a top surface 39 and a bottom surface 41. The stabilization device 21 further comprises a first arm 43 having a first inner surface 45 and a second arm 47 having a second inner surface 49. The first arm 43 and the second arm 47 each extend upwardly from the top surface 39 of the base 37. The first inner surface 45 and the second inner surface 49 define between them a space 51 for receiving the valve or sample port 27 for the catheter.

As seen in FIG. 1, the valve or sample port 27 includes inlet and outlet components 53 and 55, respectively, and an enlarged portion 57 between the inlet and outlet components. The upstream drainage line 29 connects to the inlet component 53 of the valve or sample port 27 and the downstream drainage line 31 connects to the outlet component 55 of the valve or sample port. The first inner surface 45 and the second inner surface 49 further define between them inlet and outlet openings 57 and 59 at ends of the space 51 for the inlet and outlet components of the valve or sample port 27. The inlet and outlet openings 57 and 59 are narrower than a widest portion of the space 51.

The space 51 is sized to permit the valve or sample port 27 to be positioned between the first inner surface 45 and the second inner surface 49 with its inlet component 53 extending through the inlet opening 57 of the space and its outlet component 55 extending through the outlet opening 59 of the space. The space 51 is ordinarily sized to prevent any twisting or lateral movement of the valve or drainage port 27 or of any portions of the upstream drainage line 29 or the downstream drainage line 31 proximate the stabilization device 21.

The first arm 43 and the second arm 47 may have a degree of elasticity to them such that the valve or sample port 27 can be press-fit between the first and second arms and retained in place by those arms. For example, at least one of and usually both the first arm 43 and the second arm 47 can be elastically bendable relative to the base 37 such that an original size of the space 51 can be enlarged by applying an outwardly directed force to at least one bendable one of the first arm and the second arm and, after releasing the force, the at least one of the bendable one of the first arm and the second arm will move inwardly and return the space to the original size.

The first arm 43 and the second arm 47 may be concave as shown in FIGS. 1-4, or may be substantially flat. As seen in FIGS. 2-4 (and in phantom in FIG. 1), upper ends of the first and second arms 43 and 47, or portions of the first and second arms, such as at upper corners 43′ and 47′ of the first and second arms near the inlet opening 57 and the outlet opening 57 and 59 may be shaped to curve inward relative to the space 51 to further assist in retaining the valve or sample port 27. In addition or alternatively, the valve or sample port 27 can be retained in the space 51 by an adhesive on at least one of the first inner surface 45 and the second inner surface 49.

The first arm 43 can be attached to the base 37 via a first bracket 63 and the second arm 47 can be attached to the base via a second bracket 65. The first bracket 63 and the second bracket 65 can be attached to the base 37 via one or more fasteners 67 (shown in phantom in FIG. 1). Alternatively, or in addition, the first bracket 63 and the second bracket 65 can be attached to the base 37 via adhesive or by otherwise bonding the brackets to the base. Instead of providing first and second brackets, the first arm 43, the second arm 47, and the base 37 may be integrally formed, such as by plastic molding.

The stabilization device 21 may be attached to a user's leg in a variety of suitable ways. As seen in phantom in FIG. 1, a strap 69, such as but not necessarily an elastic strap, may be attached to opposite ends 71 and 73 of the base 37, and the strap can be attached around a user's leg. Alternatively or in addition, as also shown in phantom in FIG. 1, adhesive strips 75 may be attached to opposite ends 71 and 73 of the base for attaching the device to a user's leg. Alternatively or in addition to either the strap 69 and the adhesive strips 75, an adhesive material may be applied to the bottom surface 41 of the base.

In the present application, the use of terms such as “including” is open-ended and is intended to have the same meaning as terms such as “comprising” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

While this invention has been illustrated and described in accordance with a preferred embodiment, it is recognized that variations and changes may be made therein without departing from the invention as set forth in the claims.

What is claimed is:

1. A catheter stabilization device, comprising:
a base having a top surface and a bottom surface; and
a first arm having a first inner surface and a second arm having a second inner surface, the first arm and the second arm each extending upwardly from the top surface of the base, the first inner surface and the second inner surface defining between them a space for receiving a valve or sample port for a catheter, the valve or sample port including inlet and outlet components and an enlarged portion between the inlet and outlet components, the first inner surface and the second inner surface further defining between them inlet and outlet openings at ends of the space for the inlet and outlet components of the valve or sample port, the inlet and outlet openings being narrower than a widest portion of the space.

2. The catheter stabilization device as set forth in claim 1, comprising an adhesive material on the bottom surface of the base for attaching the device to a user's leg.

3. The catheter stabilization device as set forth in claim 1, comprising a strap attached to opposite ends of the base, the strap being attachable around a user's leg.

4. The catheter stabilization device as set forth in claim 3, wherein the strap is elastic.

5. The catheter stabilization device as set forth in claim 1, comprising adhesive strips attached to opposite ends of the base for attaching the device to a user's leg.

6. The catheter stabilization device as set forth in claim 1, wherein the first arm is attached to the base via a first bracket and the second arm is attached to the base via a second bracket.

7. The catheter stabilization device as set forth in claim 1, wherein upper corners of the first arm and the second arm next to the inlet opening and the outlet opening curve inward toward the space.

8. The catheter stabilization device as set forth in claim 1, wherein the first arm, the second arm, and the base are integrally formed.

9. The catheter stabilization device as set forth in claim 1, wherein at least one of the first arm and the second arm is elastically bendable relative to the base such that an original size of the space can be enlarged by applying an outwardly directed force to at least one bendable one of the first arm and the second arm and, after releasing the force, the at least one of the bendable one of the first arm and the second arm moves inwardly and returns the space to the original size.

10. The catheter stabilization device as set forth in claim 1, comprising an adhesive on at least one of the first inner surface and the second inner surface.

11. A catheter stabilization system, comprising:
a catheter comprising a drainage port;
a valve or sample port attached to the drainage port, the valve or sample port including inlet and outlet components and an enlarged portion between the inlet and outlet components; and
a catheter stabilization device, the catheter stabilization device comprising a base having a top surface and a bottom surface, and a first arm having a first inner surface and a second arm having a second inner surface, the first arm and the second arm each extending upwardly from the top surface of the base, the first inner surface and the second inner surface defining between them a space of sufficient size to receiving the enlarged portion of the valve or sample port, the first inner surface and the second inner surface further defining between them inlet and outlet openings at ends of the space for the inlet and outlet components of the valve or sample port, the inlet and outlet openings being narrower than a widest portion of the space.

12. The catheter stabilization system as set forth in claim 11, comprising an adhesive material on the bottom surface of the base for attaching the catheter stabilization device to a user's leg.

13. The catheter stabilization system as set forth in claim 11, comprising a strap attached to opposite ends of the base, the strap being attachable around a user's leg.

14. The catheter stabilization system as set forth in claim 13, wherein the strap is elastic.

15. The catheter stabilization system as set forth in claim 11, comprising adhesive strips attached to opposite ends of the base for attaching the catheter stabilization device to a user's leg.

16. The catheter stabilization system as set forth in claim 11, wherein the first arm is attached to the base via a first bracket and the second arm is attached to the base via a second bracket.

17. The catheter stabilization system as set forth in claim 11, wherein upper corners of the first arm and the second arm next to the inlet opening and the outlet opening curve inward toward the space.

18. The catheter stabilization system as set forth in claim 11, wherein the first arm, the second arm, and the base are integrally formed.

19. The catheter stabilization system as set forth in claim 11, wherein at least one of the first arm and the second arm is elastically bendable relative to the base such that an original size of the space can be enlarged by applying an outwardly directed force to at least one bendable one of the first arm and the second arm and, after releasing the force, the at least one of the bendable one of the first arm and the second arm moves inwardly and returns the space to the original size.

20. The catheter stabilization system as set forth in claim 11, comprising an adhesive on at least one of the first inner surface and the second inner surface.
